(19) United States
(12) Patent Application Publication
(10) Pub. No.: US 2016/0130630 A1
(43) Pub. Date: May 12, 2016

(54) METHOD FOR DETECTING CAMPYLOBACTER

(71) Applicant: JNC CORPORATION, Tokyo (JP)

(72) Inventor: Hajime TERAMURA, Kanagawa (JP)

(73) Assignee: JNC CORPORATION, Tokyo (JP)

(21) Appl. No.: 14/932,986

(22) Filed: Nov. 5, 2015

(30) Foreign Application Priority Data
Nov. 6, 2014 (JP) 2014-225976

(51) Int. Cl. C12Q 1/04

(52) U.S. Cl. CPC C12Q 1/045; C12Q 1/04; G01N 2333/205

(57) ABSTRACT

A selective isolation medium that is not influenced by existence of ESBL-producing bacteria and can clearly detect Campylobacter bacteria.

Cefoperazone and Cefoxitin are incorporated into the culture medium for detecting Campylobacter bacteria.

### CROSS-REFERENCE TO RELATED APPLICATIONS

This application claims the priority benefit of Japan application serial no. 2014-225976, filed on Nov. 6, 2014. The entirety of the above-mentioned patent application is hereby incorporated by reference herein and made a part of this specification.

### TECHNICAL FIELD

The present invention relates to a culture medium that can detect Campylobacter bacteria with high selectivity, and a method for producing the same.

### BACKGROUND ART

Campylobacter jejuni (hereafter, referred to as C. jejuni) and Campylobacter coli (hereafter, referred to as C. coli) that belong to the Campylobacter genus being Gram-negative microaerophilicc bacteria are known as bacteria responsible for food poisoning. The food poisoning due to the bacteria causes not only gastroenteritis such as diarrhea but also a Guillain-Barre syndrome, and becomes severe in several cases. Such bacteria are widely distributed in intestinal tracts of livestock and poultry. Therefore, control thereof is important in view of food hygiene and safety (Non-patent literature No. 1).

In general, in stages of testing of bacteria in food, diagnosis of a patient whose bacterial infection is suspected and a drug susceptibility test for selecting an antimicrobial agent for treatment, a selective isolation medium is used to isolate objective bacteria. The selective isolation medium is a generic term for a culture medium devised such that only objective microorganisms to be isolated can grow as much as possible.

C. jejuni and C. coli are known to have resistance to cephem antibiotics. Therefore, the antibiotics have been so far used as a selective agent in isolation and detection thereof (patent literature No. 1). For example, testing is known in which a specimen is applied onto a selective isolation medium such as a modified Charcoal Cefoperazone Deoxycholate Agar (mCCDA) medium containing Cefoperazone being the cephem antibiotics, and then subjected to microaerophilic culture, and thus such bacteria are detected (Non-patent literature No. 2).

### CITATION LIST

### Patent Literature

Patent literature No. 1: JP H10-127275 A.

### Non-Patent Literature

Non-patent literature No. 1: Shokuhin Eiseikensa Shishin Biseibutsuhen (Guidelines on Food Hygiene Testing, Volume: Microorganisms), pp. 225 to 235, Jun. 30, 2004, Japan Food Hygiene Association, under the editorship of Ministry of Health, Labour and Welfare.
Non-patent literature No. 2: Pamphlet of “Campylobacter Blood-free Selective Medium (CCDA),” (http://www3 kanto.co.jp/rinsyo/pdf/101 pdf), Kanto Chemical Co., Inc.

### SUMMARY OF INVENTION

### Technical Problem

On a mCCDA medium described in Non-patent literature No. 2, growth of almost all microorganisms other than Campylobacter bacteria is suppressed by deoxycholic acid and Cefoperazone. However, bacteria that produce extended spectrum beta-lactamase (ESBL) in Klebsiella pneumoniae and Eschericia coli have recently increased to pose a problem in which the bacteria decompose kinds of cephem antibiotics in a wide range to have resistance thereto. More specifically, detection of Campylobacter bacteria becomes difficult from facts in which Cefoperazone in the mCCDA medium generally used upon detecting Campylobacter bacteria is inactivated by ESBL-producing bacteria, and ESBL-producing bacteria further rapidly and actively grow on the culture medium, in comparison with Campylobacter bacteria, to mask colony of Campylobacter bacteria.

In addition, clavulanic acid or the like is known as a β-lactamase inhibitor, but is significantly unstable and easily inactivated in the culture medium, and therefore is unsuitable for use in the selective isolation medium in view of storage stability.

Further, a general Campylobacter selective isolation medium, including the mCCDA medium, ordinarily contains activated-carbon powder in order to remove peroxide or the like that inhibits growth of Campylobacter bacteria. Therefore, the culture medium is opaque, and has a problem of difficulty in differentiating the colony of Campylobacter bacteria, the colony being formed in the form of transparent water drops.

In view of such a situation, the invention is contemplated for providing a selective isolation medium that is not influenced by existence of ESBL-producing bacteria and can clearly detect Campylobacter bacteria.

### Solution to Problem

The present inventors have diligently continued to conduct study in order to solve the problems as described above. As a result, the present inventors have focused attention on Cefoxitin being a cephamycin drug that is not decomposed by ESBL, even while Cefoxitin is a cephem antibiotic to exhibit a good growth inhibitory property against ESBL-producing bacteria, and have completed the invention.

More specifically, the invention includes the items described below.

Item 1. A culture medium for detecting Campylobacter bacteria, containing Cefoperazone and Cefoxitin.

Item 2. The culture medium for detecting Campylobacter bacteria according to item 1, further containing a color former.

Item 3. The culture medium for detecting Campylobacter bacteria according to item 1 or 2, further containing bile acid and/or salt thereof.

Item 4. A kit for preparing a culture medium for detecting Campylobacter bacteria, containing Cefoperazone, Cefoxitin, granular activated-carbon, a nutritional ingredient and a gelling agent.

Item 5. A method for producing a culture medium for detecting Campylobacter bacteria, including: a step of mixing water with a content in the kit for preparing the culture medium for detecting Campylobacter bacteria according to item 4 to obtain a mixed liquid; a step of removing the granular activated-carbon from the mixed liquid; and a step of solidifying the mixed liquid.

Item 6. A method for detecting Campylobacter bacteria, including: a step of inoculating a specimen to the culture medium for detecting Campylobacter bacteria according to any one of items 1 to 3; a step of culturing Campylobacter bacteria contained in the specimen; and a step of detecting colony of the bacteria.

### Advantageous Effects of Invention

The invention provides a transparent selective isolation medium that is not influenced by existence of ESBL-producing bacteria and can clearly detect Campylobacter bacteria.

### DESCRIPTION OF EMBODIMENTS

A culture medium for detecting Campylobacter bacteria according to the invention contains Cefoperazone and Cefoxitin as selective substances.

Cefoperazone suppresses growth of almost all microorganisms other than Campylobacter bacteria. A content of Cefoperazone in the culture medium is preferably approximately 1 to approximately 100 mg/L, and further preferably approximately 32 to approximately 64 mg/L, as a concentration during use.

Cefoxitin is not subjected to decomposition by ESBL and suppresses growth of ESBL-producing bacteria. A content of Cefoxitin in the culture medium is preferably approximately 1 to approximately 10 mg/L, and further preferably approximately 2 to approximately 6 mg/L, as a concentration during use.

Moreover, a ratio of the concentrations during use for the contents of two kinds of the selective substances in the culture medium is preferably approximately 32:1 to approximately 5:1 in terms of Cefoperazone:Cefoxitin, and further preferably approximately 8:1 in terms of Cefoperazone:Cefoxitin.

Selection and isolation of Campylobacter bacteria, particularly C. jejuni and C. coli, can be performed with high accuracy by a combination of two kinds of the selective substances. The accuracy of selection and isolation can be improved by adjusting the ratio within the range described above.

Cefoxitin is one cephamycin antibiotic in a cephem base, and is known to have wide-ranged antibacterial spectra, and has been used so far as the selective substance of Methicillin-resistant Staphylococcus aureus (MRSA).

The present inventors have found that Cefoxitin does not inhibit growth of Campylobacter bacteria and inhibits the growth of ESBL-producing bacteria, and have arrived at structure of the culture medium of the invention.

In addition, Cefotetan classified in the cephamycin base in a manner similar to Cefoxitin is not decomposed by ESBL. However, Cefotetan inhibits the growth of Campylobacter bacteria, and therefore is unsuitable for selective isolation of Campylobacter bacteria.

The culture medium for detecting Campylobacter bacteria according to the invention further preferably contains a color former. The former is applied for forming colored colony in selectively isolated Campylobacter bacteria to facilitate detection thereof.

The color former is an oxidation-reduction indicator from which a colored chromogen compound is ordinarily released in an electron transfer system in a respiratory reaction of bacteria. Specific examples of such an oxidation-reduction indicator preferably include tetrazolium violet, 2,3,5-triphenyl tetrazolium chloride, p-iodonitrotetrazolium violet, p-nitro blue tetrazolium chloride, nitro blue tetrazolium chloride and 3-(4,5-dimethyl-2-thiazolyl)-2,5-diphenyl-2H-tetrazolium bromide. Among the color formers, tetrazolium violet is further preferred due to good coloring of the colony and only limited influence on the growth of Campylobacter bacteria.

A content of the color former in the culture medium is preferably approximately 0.005 to approximately 0.5 mg/L, and further preferably approximately 0.01 to approximately 0.25 mg/L, as a concentration during use.

The culture medium for detecting Campylobacter bacteria according to the invention ordinarily further contains a gelling agent. The gelling agent holds moisture in the culture medium to support the culture medium and form a solid medium, and thus can facilitate operation of a method for detecting objective bacteria as described later.

Specific examples of the gelling agent include agar, polyvinyl alcohol and a cellulose derivative such as methylcellulose, carboxymethylcellulose and hydroxyalkylcellulose; starch and a derivative thereof; polysaccharide such as hyaluronic acid, guar gum and xanthan gum; an acrylic acid derivative such as polyacrylic acid, polyacrylate and an acrylic acid-vinyl alcohol copolymer; polyether such as polyethylene glycol and polypropylene glycol; and protein such as collagen, but are not particularly limited thereto.

Moreover, a content thereof can be arbitrarily adjusted in an amount ordinarily used for the solid medium.

Moreover, the culture medium of the invention may be formed into an aspect of ordinary gel-form and also a sheet-form culture medium (WO 97/24432 A or the like).

The culture medium for detecting Campylobacter bacteria according to the invention may further contain bile acid and/or salt thereof. The bile acid suppresses growth of Gram-positive bacteria.

Specific examples of the bile acid preferably include cholic acid and deoxycholic acid, and also preferably include alkali metal salt such as sodium salt and potassium salt.

A content of the bile acid and/or the salt thereof in the culture medium is preferably approximately 0.5 to approximately 5 mg/L, and further preferably approximately 1 to approximately 2 g/L, as a concentration during use.

The culture medium for detecting Campylobacter bacteria according to the invention can arbitrarily contain, in addition to the ingredients described above, an ingredient ordinarily used in the culture medium for microorganisms, such as water, other selective substances, a nutritional ingredient, inorganic salts and a pH adjuster.

Specific examples of other selective substances preferably include an antibacterial compound such as vancomycin and amphotericin B in order to suppress growth of Gram-positive bacteria and eumycetes.

Specific examples of the nutritional ingredient preferably include peptone, soy peptone, a yeast extract, an animal meat extract, a fish meat extract, glucose, sucrose and lactose.

Specific examples of the inorganic salts preferably include inorganic acid metal salt such as sodium chloride and ferrous sulfate, and organic acid metal salt such as sodium pyruvate.

In the culture medium for detecting Campylobacter bacteria according to the invention, from a viewpoint of the growth of Campylobacter bacteria, pH during use is preferably approximately 7.0 or more, and further preferably approximately 7.0 to approximately 8.0.

Moreover, the culture medium for detecting Campylobacter bacteria according to the invention is not particularly limited, but preferably substantially contains no blood. The reason is that the culture medium substantially containing no blood has no turbidity, and therefore the colony in the form of transparent water drops, the colony being formed by Campylobacter bacteria, can be clearly differentiated and detected. “Substantially containing no blood” means that the content is approximately 0.0001% by weight or less based on the total amount of the culture medium.

A material and equipment for culturing the microorganisms and the culture medium for the microorganisms according to the invention are not particularly limited, but can be prepared by procedures described below, for example.

The culture medium is prepared according to a step of mixing, with water, Cefoperazone, Cefoxitin, granular activated-carbon, a nutritional ingredient and a gelling agent to obtain a mixed liquid, a step of removing the granular activated-carbon from the mixed liquid and a step of solidifying the mixed liquid.

Here, the step of obtaining the mixed liquid is performed while stirring is ordinarily performed, warming may be performed, and sterilization is preferably performed in an autoclave or the like. Moreover, Cefoperazone and Cefoxitin have weak resistance to heat. Therefore, the mixed liquid is heated and then cooled, and then Cefoperazone and Cefoxitin are preferably added thereto and mixed therein. Moreover, Cefoperazone, Cefoxitin, the nutritional ingredient and the gelling agent are preferably sufficiently dissolved into water. Moreover, other arbitrary ingredients for the culture media can also be added during a suitable step.

Moreover, the step of removing the granular activated-carbon may be performed by spontaneously precipitating the carbon by allowing the liquid to stand or by precipitating the carbon by centrifugal separation to separate and obtain a supernatant only, or the granular activated-carbon may be removed by filtration with a filter or the like, but the operation of spontaneous precipitation by allowing the liquid to stand is simple and preferred.

In addition, Cefoperazone, Cefoxitin, the granular activated-carbon, the nutritional ingredient and the gelling agent can be formed into a kit for preparing the culture medium according to the invention.

Moreover, the granular activated-carbon is a material for removing peroxide or the like that inhibits the growth of Campylobacter bacteria from the culture medium. However, in the method for preparing the culture medium according to the invention, a finished culture medium can be made transparent by removing the granular activated-carbon on the way of preparation. Thus, the colony of Campylobacter bacteria, the colony being formed in the form of the transparent water drops, can be clearly and easily differentiated.

A mean particle size of the granular activated-carbon is preferably approximately 1 to approximately 10 millimeters, and further preferably approximately 2.5 to approximately 5 millimeters. Moreover, an amount of mixing the granular activated-carbon is preferably approximately 20 to approximately 100 g/L, and further preferably approximately 30 to approximately 50 g/L, in the mixed liquid. Moreover, the granular activated-carbon is ordinarily preferably mixed for approximately 15 minutes or more in the mixed liquid from a viewpoint of removal of the peroxide or the like.

The culture medium for detecting Campylobacter bacteria according to the invention can be preferably utilized for a method for detecting Campylobacter bacteria in the specimen. The method includes a step of inoculating the specimen to the culture medium of the invention, a step of culturing Campylobacter bacteria contained in the specimen and a step of detecting colony of microorganisms. As conditions in the culturing step, the conditions of approximately 33 to approximately 45° C., and approximately 24 to approximately 48 hours under microaerophilic conditions are preferred.

The culture medium of the invention can suppress the growth of other microorganisms including mixed ESBL bacteria, and is transparent. Therefore, Campylobacter bacteria can be easily detected according to the detection method of the invention.

As detection objective bacteria that can be selectively isolated by the culture medium according to the invention, C. jejuni and C. coli are particularly preferred among Campylobacter bacteria.

Specific examples of the specimen that is applied to the detection method according to the invention include perishable foods such as meat, and fish and shellfishes, a clinical specimen such as feces, sea water and a wiping specimen in a cooking place and a hospital. Moreover, a culture fluid obtained by preculturing the specimens in Tryptic Soy Broth and Preston Broth or the like, and a culture fluid obtained by further culturing the culture fluid in a culture medium for proliferating microbial cells can also be used as the specimens.

It will be apparent to those skilled in the art that various modifications and variations can be made in the invention and specific examples provided herein without departing from the spirit or scope of the invention. Thus, it is intended that the invention covers the modifications and variations of this invention that come within the scope of any claims and their equivalents.

The following examples are for illustrative purposes only and are not intended, nor should they be interpreted to, limit the scope of the invention.

### Example

The invention will be described in greater detail by way of Examples. The invention is not limited by the Examples.

### (1) Preparation of Culture Medium

Base culture media having formulations shown in Table 1 were mixed, and the resulting mixture was dissolved with each other at 121° C. for 15 minutes, and then the resulting mixture was cooled to approximately 50° C. An aqueous solution of a color former and a selective substance having formulations shown in Table 2 was added thereto, and the resulting mixture was well mixed, and then was allowed to stand for 5 minutes. Then, a supernatant portion of the culture medium from which granulated activated-carbon was precipitated was dispensed by 20 mL to plastic petri dishes (diameter: 90 mmφ), respectively. Then, the plastic petri dishes were allowed to stand until the culture media were solidified to prepare the culture media of the invention. In addition, the culture media of the invention are transparent.

As a comparative culture medium, a CODA agar medium (Non-patent literature No. 2, made by Oxoid Limited) containing Cefoperazone and amphotericin B as selective substances without containing Cefoxitin was used. In addition, appearance of the CODA agar medium is opaque and black due to carbon powder.

As a positive control, a commercially available sheep blood agar (BA) medium (made by Nissui Pharmaceutical Co., Ltd.) was used.

TABLE 1

| | (g) |
|---|---|
| Casein peptone | 10 |
| Meat peptone | 10 |
| Sodium chloride | 5 |
| Casamino acid | 3 |
| Sodium deoxycholate | 1 |
| Ferrous sulfate | 0.25 |
| Sodium pyruvate | 0.25 |
| Agar | 15 |
| Granulated activated-carbon | 40 |
| (mean particle size: 2.5 mm) | |
| Purified water | 1,000 |

pH7.4 ± 0.2

TABLE 2

| | |
|---|---|
| Tetrazolium violet | 0.01 g |
| Cefoperazon sodium salt | 32 mg |
| Cefoxitin sodium salt | 4 mg |
| Purified water | 10 mL |

### (2) Provision of Strain for Testing

Campylobacter jejuni, Campylobacter coli, Proteus mirabilis, Pseudomonas aeruginosa, Bacillus subtilis, Enterococcus faecalis and Escherichia coli were provided as test strains. Among the strains, as C. jejuni and C. coli, strains obtained by being precultured on BA media for 24 hours under microaerophilic conditions, and as other strains, strains obtained by being precultured on BA media for 24 hours under aerobic conditions were provided for testing, respectively. Each test strain was suspended into a 0.05% agar-added sterile physiological saline using a sterile cotton swab so as to correspond to McFarland nephelometry No. 1 (approximate 3.0×10⁸ CFU/mL), and taken as a bacteria stock solution. Then, each bacteria stock solution was repeatedly subjected to 10-fold serial dilution to 10⁻⁷ by using the 0.05% agar-added sterile physiological saline, and each bacteria solution was provided for testing according to the Miles and Misra method (Shin Saikinbaichigaku Koza (New Bacterial Culture Medium Science Course)-Jo (First)-(second edition), pp. 182 to 192, Kabushikigaisha Kindaishuppan, 1986). All culture media provided for testing were cultured at 42° C. for 48 hours under microaerophilic culture, and then the number of grown bacteria and color tone of colony were confirmed.

The results of the number of grown bacteria are shown in Table 3.

TABLE 3

| | | Culture | | |
|---|---|---|---|---|
| | Dilution | media of the | | |
| | level | invention | CCDA media | BA media |
| Campylobacter jejuni | 10⁻² | + | + | + |
| ATCC33560 | 10⁻³ | + | + | + |
| | 10⁻⁴ | + | + | + |
| | 10⁻⁵ | + | + | + |
| | 10⁻⁶ | 34 | 30 | 48 |
| | 10⁻⁷ | − | 2 | 10 |
| Campylobacter coli | 10⁻² | + | + | + |
| ATCC33559 | 10⁻³ | + | + | + |
| | 10⁻⁴ | + | + | + |
| | 10⁻⁵ | + | + | + |
| | 10⁻⁶ | + | + | + |
| | 10⁻⁷ | 18 | 23 | 13 |
| Proteus mirabilis | 10⁻² | − | − | + |
| ATCC29906 | 10⁻³ | − | − | + |
| | 10⁻⁴ | − | − | + |
| | 10⁻⁵ | − | − | + |
| | 10⁻⁶ | − | − | + |
| | 10⁻⁷ | − | − | 11 |
| Pseudomonas | 10⁻² | 1 | 9 | + |
| aeruginosa | 10⁻³ | − | − | + |
| ATCC9027 | 10⁻⁴ | − | − | + |
| | 10⁻⁵ | − | − | + |
| | 10⁻⁶ | − | − | + |
| | 10⁻⁷ | − | − | 19 |
| Bacillus subtilis | 10⁻² | − | − | + |
| ATCC6633 | 10⁻³ | − | − | + |
| | 10⁻⁴ | − | − | + |
| | 10⁻⁵ | − | − | + |
| | 10⁻⁶ | − | − | 17 |
| | 10⁻⁷ | − | − | 4 |
| Enterococcus faecalis | 10⁻² | − | − | + |
| ATCC19433 | 10⁻³ | − | − | + |
| | 10⁻⁴ | − | − | + |
| | 10⁻⁵ | − | − | + |
| | 10⁻⁶ | − | − | 40 |
| | 10⁻⁷ | − | − | 3 |
| Escherichia coli | 10⁻² | 2 | + | + |
| (Feces isolate 1; | 10⁻³ | − | + | + |
| ESBL-producing | 10⁻⁴ | − | + | + |
| bacteria) | 10⁻⁵ | − | + | + |
| | 10⁻⁶ | − | + | + |
| | 10⁻⁷ | − | 28 | 30 |
| Escherichia coli | 10⁻² | − | + | + |
| (Feces isolate 2; | 10⁻³ | − | + | + |
| ESBL-producing | 10⁻⁴ | − | + | + |
| bacteria) | 10⁻⁵ | − | + | + |
| | 10⁻⁶ | − | 35 | 36 |
| | 10⁻⁷ | − | 4 | 8 |

In the culture media of the invention, detection of C. jejuni and C. coli as purple colored colony was clearly allowed, and suppression of growth of other bacteria including ESBL-producing bacteria was satisfactorily allowed. On the other hand, in the CCDA culture medium, the transparent colony of C. jejuni and C. coli was hard to observe, and no suppression of the growth of ESBL-producing bacteria was allowed.

Although the invention has been described and illustrated with a certain degree of particularity, it is understood that the disclosure has been made only by way of example, and that numerous changes in the conditions and order of steps can be resorted to by those skilled in the art without departing from the spirit and scope of the invention.

### INDUSTRIAL APPLICABILITY

The present invention provides a transparent selective isolation medium that is not influenced by existence of ESBL-producing bacteria and can clearly detect Campylobacter bacteria, and therefore is industrially significantly useful.

What is claimed is:

1. A method for detecting Campylobacter bacteria, comprising:
a step of inoculating a specimen to a culture medium comprising Cefoperazone and Cefoxitin;
a step of culturing Campylobacter bacteria contained in the specimen; and
a step of detecting colony of Campylobacter bacteria.

2. The method according to claim 1, wherein the culture medium further comprises a color former.

3. The method according to claim 1, wherein the culture medium further comprises bile acid, salt of bile acid, or combination thereof.

4. A method for producing a culture medium for detecting Campylobacter bacteria, comprising:
a step of mixing water with Cefoperazone, Cefoxitin, granular activated-carbon, a nutritional ingredient and a gelling agent to obtain a mixed liquid;
a step of removing the granular activated-carbon from the mixed liquid;
and a step of solidifying the mixed liquid.
